(19) United States
(12) Patent Application Publication
(10) Pub. No.: US 2008/0051881 A1
(43) Pub. Date: Feb. 28, 2008

(54) Medical devices comprising porous layers for the release of therapeutic agents

(76) Inventors: James Q. Feng, Maple Grove, MN (US); Jan Weber, Maple Grove, MN (US); Liliana Atanasoska, Edina, MN (US)

Correspondence Address: MAYER & WILLIAMS PC, 251 NORTH AVENUE WEST, 2ND FLOOR, WESTFIELD, NJ, 07090, US

(21) Appl. No.: 11/789,983

(22) Filed: Apr. 26, 2007

Related U.S. Application Data
(60) Provisional application No. 60/839,751, filed on Aug. 24, 2006.

(51) Int. Cl. A61F 2/82

(52) U.S. Cl. 623/1.39; 623/1.42; 623/1.49; 977/906

(57) ABSTRACT

In accordance with an aspect of the invention, implantable or insertable medical devices are provided in which a porous layer is disposed over a therapeutic-agent-containing region. In accordance with another aspect of the invention, medical devices are fabricated by a method in which a porous layer is deposited over a therapeutic-agent-containing region using a field-injection-based electrospray technique.

### STATEMENT OF RELATED APPLICATIONS

This application claims the benefit of U.S. Provisional Patent Application Ser. No. 60/839,751, filed Aug. 24, 2006, entitled “Medical Devices Comprising Porous Layers For The Release Of Therapeutic Agents”, which is incorporated by reference herein.

### FIELD OF THE INVENTION

The present invention relates to medical devices which comprise a porous layer for the release of therapeutic agents.

### BACKGROUND OF THE INVENTION

The in vivo delivery of therapeutic agents within the body of a patient is common in the practice of modern medicine. In vivo delivery of therapeutic agents is often implemented using medical devices that may be temporarily or permanently placed at a target site within the body. These medical devices can be maintained, as required, at their target sites for short or prolonged periods of time, delivering biologically active agents at the target site.

In accordance with certain delivery strategies, a therapeutic agent is provided within or beneath a biostable or bioresorbable polymeric layer that is associated with a medical device. Once the medical device is placed at the desired location within a patient, the therapeutic agent is released from the medical device with a profile that is dependent, for example, upon the nature of the therapeutic agent and of the polymeric layer, among other factors.

Examples of such devices include drug eluting coronary stents, which are commercially available from Boston Scientific Corp. (TAXUS), Johnson & Johnson (CYPHER), and others. For example, the TAXUS stent contains a non-porous polymeric coating consisting of an antiproliferative drug (paclitaxel) within a biostable polymer matrix. The drug diffuses out of the coating over time. Due to the relatively low permeability of paclitaxel within the polymer matrix and due to the fact that the polymer matrix is biostable, a residual amount of the drug remains in the device beyond its period of usefulness (e.g., after the coating is overgrown with cells). Moreover, smooth surfaces by their nature do not allow for cell in-growth, and they commonly exhibit inferior cell adhesion and growth relative to textured surfaces.

### SUMMARY OF THE INVENTION

In accordance with an aspect of the invention, medical devices are provided in which a porous layer is disposed over a therapeutic-agent-containing region.

In accordance with another aspect of the invention, medical devices are fabricated by a method in which a porous layer is deposited over a therapeutic-agent-containing region using a field-injection-based electrospray technique.

Depending on the embodiment that is practiced, advantages of the present invention may include one or more of the following, among others: (a) reduced retention of therapeutic agent, (b) improved cell adhesion, (c) improved cell proliferation, (d) improved cell in-growth, (e) prevention of contact between bodily tissue and bioadverse substrates, if present, and (f) prevention of fragmentation of biodegradable substrates, if present.

These and other embodiments and advantages of the present invention will become immediately apparent to those of ordinary skill in the art upon review of the Detailed Description and Claims to follow.

### BRIEF DESCRIPTION OF THE DRAWINGS

FIG. 1 contains micrographs of prior art porous polymeric layers.

FIG. 2 is a schematic perspective view of a stent, in accordance with the invention.

FIGS. 3A-3D are schematic cross-sectional views taken along line a—a of FIG. 2, in accordance with four alternative embodiments of the present invention.

FIG. 4 is a schematic perspective view of a tubular medical device, in accordance with the invention.

FIGS. 4B-4D are schematic cross-sectional views taken along line b—b of FIG. 4A, in accordance with various alternative embodiments of the present invention.

FIGS. 5A-5E are schematic illustrations of various options that may be employed for the outer regions of FIGS. 4B and 4D, in accordance with various embodiments of the invention.

FIGS. 6A-6E are schematic illustrations of various options that may be employed for the inner regions of FIGS. 4C and 4D, in accordance with various embodiments of the invention.

### DETAILED DESCRIPTION OF THE INVENTION

In accordance with an aspect of the invention, implantable or insertable medical devices are provided in which a porous layer is disposed over a therapeutic-agent-containing region. One advantage of the porous layer is that, upon implantation or insertion of the device, therapeutic agent can diffuse through fluid (e.g., bodily fluid) within the pores of the porous layer, rather than having to diffuse though the solid material making up the porous layer (which is commonly the case with non-porous layers). This may dramatically increase release rates relative to non-porous surfaces in some embodiments. Moreover, in some embodiments of the invention, porous surfaces are provided, which promote attachment, proliferation and/or in-growth of cells (e.g., endothelial cells). In still other embodiments, porous surfaces may act as physical barriers between an underlying substrate and an outside environment, for example, segregating a bioadverse substrate and/or retaining fragments of a substrate as it is biodegraded in vivo. As used herein, a “bioadverse” substrate is one that, if not isolated in some fashion (e.g., with a porous layer in accordance with the invention), causes a biologically undesirable outcome upon implantation or insertion into a subject. An example of a substrate that is bioadverse for vascular applications is one having a material or surface chemistry or surface topology or combination thereof that causes activation of blood coagulation pathways and thrombus formation.

Medical devices benefiting from the present invention vary widely and include implantable or insertable medical devices such as, for example, catheters (e.g., renal or vascular catheters such as balloon catheters and various central venous catheters), guide wires, balloons, filters (e.g., vena cava filters and mesh filters for distil protection devices), stents (including coronary vascular stents, peripheral vascular stents, cerebral, urethral, ureteral, biliary, tracheal, bronchial, gastrointestinal and esophageal stents), stent coverings, stent grafts, vascular grafts, abdominal aortic aneurysm (AAA) devices (e.g., AAA stents, AAA grafts), vascular access ports, dialysis ports, embolization devices including cerebral aneurysm filler coils (including Guglilmi detachable coils and metal coils), embolic agents, hermetic sealants, septal defect closure devices, myocardial plugs, patches, pacemakers, lead coatings including coatings for pacemaker leads, defibrillation leads and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, among other medical devices that are implanted or inserted into the body and from which therapeutic agent is released.

Examples of medical devices further include, sutures, suture anchors, tissue staples and ligating clips at surgical sites, cannulae, metal wire ligatures, urethral slings, hernia “meshes”, artificial ligaments, orthopedic prosthesis such as bone grafts, bone plates, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, and guided-tissue-regeneration membrane films following periodontal surgery.

In various embodiments of the invention, the porous layer lies over a substrate region, and a biodegradable material lies beneath the porous layer, which biodegradable material acts to regulate the release of the therapeutic agent from the medical device into a subject upon implantation or insertion of the device into said subject.

Depending on the embodiment, the porous layers of the present invention may be biostable or biodegradable. As defined herein, a “biostable” region is one which remains intact over the time period that the medical device is intended to remain implanted within the body. Similarly, as defined herein, a “biodegradable” region is one which does not remain intact over the period which the medical device is intended to remain within the body, for example, due to any of a variety of mechanisms including dissolution, chemical breakdown, and so forth, of the region. Depending upon the device within which the biodegradable region is disposed and the mechanism of degradation of the biodegradable region this period may vary, for example, from less than or equal to 1 hour to 3 hours to 12 hours to 1 day to 3 days to 1 week to 1 month to 3 months to 1 year or longer.

Materials for forming the porous layers include the following, among others: (a) organic materials (i.e., materials containing one or more organic species), such as polymeric and non-polymeric organic materials, (b) inorganic materials (i.e., materials containing one or more inorganic species), such as metallic materials (e.g., metals and metal alloys) and non-metallic materials (e.g., carbon, semiconductors, glasses and ceramics containing various metal- and non-metal-oxides, various metal- and non-metal-nitrides, various metal- and non-metal-carbides, various metal- and non-metal-borides, various metal- and non-metal-phosphates, and various metal- and non-metal-sulfides, among others), and (c) organic-inorganic hybrids (e.g., polymer-ceramic composites, among others).

Specific examples of non-metallic inorganic materials may be selected, for example, from materials containing one or more of the following: metal oxides, including aluminum oxides and transition metal oxides (e.g., oxides of titanium, zirconium, hafnium, tantalum, molybdenum, tungsten, rhenium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon and carbon-based, ceramic-like materials such as carbon nitrides, among many others.

In this regard, certain ceramics have been shown to be bioactive. As defined herein, a “bioactive” material is a material that promotes good tissue adhesion and/or growth, for example, bone tissue or soft tissue, with minimal adverse biological effects (e.g., the formation of undesirable connective tissue such as undesirable fibrous connective tissue). Examples of bioactive ceramic materials, sometimes referred to as “bioceramics,” include calcium phosphate ceramics, for example, hydroxyapatite; calcium-phosphate glasses, sometimes referred to as glass ceramics, for example, bioglass; and metal oxide ceramics, for example, alumina and titania, among others. Metal oxide bioactivity has been also been shown to depend upon surface topography. See, e.g., Viitala R. et al., “Surface properties of in vitro bioactive and non-bioactive sol-gel derived materials,” Biomaterials, 2002 August; 23(15): 3073-86.

Specific examples of metallic inorganic materials may be selected, for example, from substantially pure metals (e.g., biostable metals such as gold, platinum, palladium, iridium, osmium, rhodium, titanium, tantalum, tungsten, and ruthenium, and bioresorbable metals such as magnesium and iron), metal alloys comprising iron and chromium (e.g., stainless steels, including platinum-enriched radiopaque stainless steel), alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), alloys comprising nickel and chromium (e.g., inconel alloys), and bioabsorbable metal alloys such as magnesium alloys and iron alloys (including their combinations with Ce, Ca, Zn, Zr, Li, etc.), among many others.

Specific examples of organic materials include polymers and other organic materials, which may be, for example, naturally occurring or synthetic, biostable or biodegradable, and may be selected, for example, from the following, among others: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen (e.g., collagen IV or V), fibronectin, elastin, chitosan, gelatin, starch, glycosaminoglycans such as hyaluronic acid; as well as blends and further copolymers of the above.

Examples of biodegradable polymers, not necessarily exclusive of those set forth above, may be selected from suitable members of the following, among many others: (a) polyester homopolymers and copolymers such as polyglycolide, poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide), poly(lactide-co-delta-valerolactone), poly(lactide-co-epsilon-caprolactone), poly(L-lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], and poly(sebacic acid-co-fumaric acid), among others (b) polyanhydride homopolymers and copolymers such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others; (c) poly(ortho esters) such as those synthesized by copolymerization of various diketene acetals and diols, and (d) amino acid based homopolymers and copolymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine, among others), and leucine and lysine-based polyester-amides; specific examples of tyrosine based polymers include poly(desaminotyrosyl-tyrosine ethyl ester adipate) or poly(DTE adipate), poly(desaminotyrosyl-tyrosine hexyl ester succinate) or poly(DTH succinate), poly(desaminotyrosyl-tyrosine ethyl ester carbonate) or poly(DTE carbonate), poly(desaminotyrosyl-tyrosine butyl ester carbonate) or poly(DTB carbonate), poly(desaminotyrosyl-tyrosine hexyl ester carbonate) or poly(DTH carbonate), and poly(desaminotyrosyl-tyrosine octyl ester carbonate) or poly(DTO carbonate), among others.

The porous layer may be, for example, porous as applied, or it may initially be non-porous, but rendered porous prior to insertion/implantation (e.g., prior to packaging), or it may become porous at a specific time after insertion/implantation.

For example, in some embodiments, the porous layer is a fibrous layer. Porous fibrous layers may be formed using any suitable fiber-based fabrication technique including, for example, various woven and non-woven techniques (e.g., knitting, braiding, winding, wrapping, spraying, fusion of short fiber segments, etc.). Examples of non-woven techniques include those that utilize thermal fusion, fusion due to removal of residual solvent, mechanical entanglement, chemical binding, and adhesive binding, among others.

Fibrous layers may be formed, for example, from pre-formed fibers (e.g., preformed metallic fibers, preformed ceramic fibers, and preformed polymer-inorganic hybrid fibers, among others) using various woven and non-woven techniques. Examples of metallic fibers include stainless steel and nitinol fibers, among others. Examples of ceramic fibers include Nextel™ fibers (aluminum oxide 62%, boron oxide 14%, silicon dioxide 24%) commercially available from 3M, MN, USA, among others. Examples of polymeric fibers include SIBS, ethylene-vinyl acetate (EVA), and polyethylene oxide (PEO) fibers. One example of a polymer-inorganic hybrid fiber is SIBS containing 1% by weight single wall carbon nanotubes. Other examples include polymer-ceramic hybrid fibers such as polymer-silica hybrid fibers and polymer-metal oxide hybrid fibers, among others.

Fibers may also be created at the time of porous layer formation. For instance, fibers for the practice of the invention may be made by any suitable fiber forming technique, including, for example, melt spinning and solvent spinning (e.g., dry spinning and wet spinning) of polymer fibers. These processes typically employ extrusion nozzles having one or more orifices, also called distributors, jets, or spinnerets. Fibers having a variety of cross-sectional shapes may be formed, depending upon the shape of the orifice(s). Some examples of fiber cross-sections include polygonal (e.g., triangular, rectangular, hexagonal, etc.), circular, oval, multi-lobed, and annular (hollow) cross-sections, among others. In melt spinning, polymers are heated to melt temperature prior to extrusion. In wet and dry spinning polymers are dissolved in a solvent prior to extrusion. In dry spinning, the extrudate is subjected to conditions whereby the solvent is evaporated, for example, by exposure to a vacuum or heated atmosphere (e.g., air) which removes the solvent by evaporation. In wet spinning the spinneret is immersed in a liquid, and as the extrudate emerges into the liquid, it solidifies. Regardless of the technique, the resulting fiber is generally taken up on a rotating mandrel or another take-up device. During take up, the fiber may be stretched (i.e., drawn) to orient the polymer molecules. A common aspect to various spinning techniques, including those described above, is that a polymer containing liquid is extruded and ultimately solidified (e.g., due to cooling, solvent removal, chemical reaction, etc.)

One particular method for forming porous tubular three-dimensional structures is described in U.S. Pat. No. 4,475,972, the disclosure of which is hereby incorporated by reference, in which these articles are made by a procedure in which fibers are wound on a mandrel and overlying fiber portions are simultaneously bonded with underlying fiber portions.

For instance, a polymer solution (or melt) can be extruded from a spinneret, thereby forming a plurality of filaments which are wound onto a rotating mandrel, as the spinneret reciprocates relative to the mandrel. The drying (or cooling) parameters may be controlled such that some residual solvent (or tackiness) remains in the filaments as they are wrapped upon the mandrel. Upon further solvent evaporation (or cooling), the overlapping fibers on the mandrel become bonded to each other.

In certain embodiments of the invention, electrostatic spinning processes may be employed. Electrostatic spinning processes have been described, for example, in Annis et al. in “An Elastomeric Vascular Prosthesis”, Trans. Am. Soc. Artif. Intern. Organs, Vol. XXIV, pages 209-214 (1978), U.S. Pat. No. 4,044,404 to Martin et al., U.S. Pat. No. 4,842,505 to Annis et al., U.S. Pat. No. 4,738,740 to Pinchuk et al., and U.S. Pat. No. 4,743,252 to Martin Jr. et al. In electrostatic spinning, electrostatic charge generation components are employed to develop an electrostatic charge between the distributor (e.g., the spinneret) and a takeup device such as a rotating mandrel. For example, the mandrel may be grounded or negatively charged, while the distributor is positively charged. Alternatively, the distributor may be grounded or negatively charged, while the mandrel can be positively charged. The potential that is employed may be constant or variable. As a result of the electrostatic charge that is generated, the polymeric fibers experience a force that accelerates them from the distributor to the mandrel. Moreover, contact between the fibers may be enhanced, because the fibers are electrostatically drawn onto the mandrel, in some instances causing the fibers to sink to some extent into underlying fibers.

Other processes whereby porous layers, including fibrous and non-fibrous layers, may be formed are electrospray processes which are based on field injection. By way of background, it is known that molecules can lose electrons (and thus become positively charged) when placed in a very high electric field. High fields can be created by applying a high voltage between a cathode and an anode referred to as a field emitter, which typically contains one or more sharpened points which result in high electric fields.

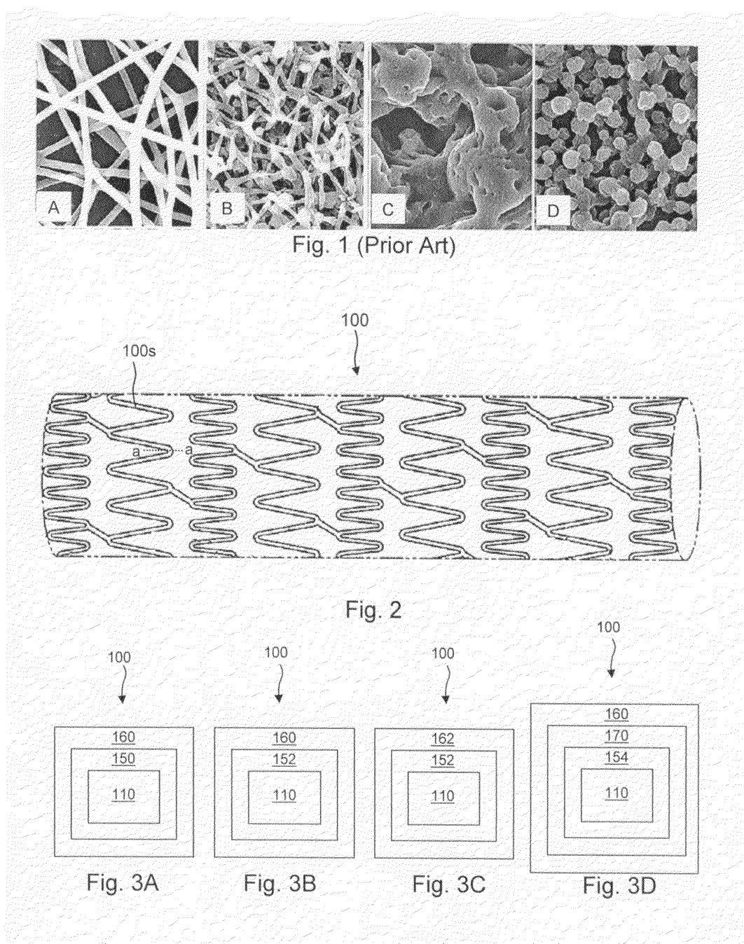

Flow-limited field-injection electrostatic spraying (FFESS) is one example of a field-injection-based electrospray technique. FFESS gives excellent control of the morphology of a deposited material. In one known FFESS technique, charge injection is achieved using a nano-sharpened metallic needle positioned within a smooth glass capillary nozzle. This technique produces sprays that are finer and more controllable than sprays produced by conventional electrospraying techniques, which typically employ hypodermic needles as the spray nozzle, the reason being that the charge transfer is more effective in the FFESS method. By varying parameters such as applied voltage, solvent properties such vapor pressure, and polymer solution properties such as flow rate, surface tension, dielectric constant, polymer concentration and viscosity, porous layers having a variety of deposited morphologies can be produced including fibrous layers such as interconnected fibrous layers, interconnected particles such as melded spheres, and so forth. In this regard, see, e.g., C. Berkland et al., “Controlling surface nano-structure using flow-limited field-injection electrostatic spraying (FFESS) of poly(d,l-lactide-co-glycolide),” Biomaterials 25 (2004) 5649-5658. Some of the porous layers formed by Berkland et al., specifically, two interconnected fibrous layers and two interconnected particulate layers, are shown in micrographs A, B, C and D of FIG. 1. While the polymers used in the techniques described in Berkland et al. are biodegradable polymers, specifically, poly(d,l-lactide-co-glycolide), field-injection-based electrospray techniques, including FFESS, are not so limited, but rather are applicable to a broad range of polymeric materials. Id.

Using the above and other techniques, a wide variety of porous layers, including interconnected fibrous layers and interconnected particle layers, may be formed. Fiber and particle diameter within such porous layers can vary widely in size, but are typically less than 50 microns (μm), for example, ranging from 50 microns to 25 microns to 10 microns to 5 microns to 2.5 microns to 1 micron to 0.5 micron (500 nm) to 0.25 micron (250 nm) to 0.1 micron (100 nm) to 0.05 micron (50 nm) to 0.02 micron (20 nm), or less.

In other embodiments of the invention, hybrid polymer-ceramic porous regions are formed in conjunction with sol-gel-based processing. By way of background, it is well known that ceramic regions may be formed using sol-gel processing. In a typical sol-gel process, precursor materials, typically selected from inorganic metallic and semi-metallic salts, metallic and semi-metallic complexes/chelates, metallic and semi-metallic hydroxides, and organometallic and organo-semi-metallic compounds such as metal alkoxides and alkoxysilanes, are subjected to hydrolysis and condensation (also referred to sometimes as polymerization) reactions, thereby forming a “sol” (i.e., a suspension of solid particles within a liquid). For example, an alkoxide of choice (such as a methoxide, ethoxide, isopropoxide, tert-butoxide, etc.) of a semi-metal or metal of choice (such as silicon, germanium aluminum, zirconium, titanium, tin, iron, hafnium, tantalum, molybdenum, tungsten, rhenium, iridium, etc.) may be dissolved in a suitable solvent, for example, in one or more alcohols. Subsequently, water or another aqueous solution such as an acidic or basic aqueous solution (which aqueous solution can further contain organic solvent species such as alcohols) is added, causing hydrolysis and condensation to occur. Further processing of the sol enables solid materials to be made in a variety of different forms. For instance, “wet gel” coatings can be produced by spray coating, coating with an applicator (e.g., by roller or brush), ink-jet printing, screen printing, and so forth. The wet gel is then dried to form a ceramic region. Further information concerning sol-gel materials can be found, for example, in Viitala R. et al., “Surface properties of in vitro bioactive and non-bioactive sol-gel derived materials,” Biomaterials, 2002 August; 23(15):3073-86.

Polymer-ceramic composite (hybrid) regions may be formed based upon analogous processes, as well as upon principles of polymer synthesis, manipulation, processing, and so forth. Sol gel processes are suitable for use in conjunction with polymers and their precursors, for example, because they can be performed at ambient temperatures. A review of various techniques for generating polymeric-ceramic composites can be found, for example, in G. Kickelbick, “Concepts for the incorporation of inorganic building blocks into organic polymers on a nanoscale” Prog. Polym. Sci., 28 (2003) 83-114.

It is known, for example, to impregnate a gel such as a xerogel with monomer and polymerize the monomer within the gel. Best results are obtained where interactions between the monomer/polymer and the gel are sufficiently strong to prevent macroscopic phase separation. Conversely, it is also known, for example, to generate polymeric-ceramic composites by conducting sol gel processing in the presence of a preformed polymer, which techniques tend to be successful, for example, where the polymer is soluble in the sol-forming solution and/or where the polymer has substantial interactions with the ceramic phase (e.g., due to hydrogen bonding between hydroxyl groups and electronegative atoms within the polymeric and ceramic phases, etc.), which prevent macroscopic phase separation. One way of improving the interactions between the polymeric and ceramic components is to employ a charged polymer, or ionomer. For this purpose, polymers may be functionalized with anionic groups, such as sulfonate or carboxylate groups, among others, or cationic groups, such as ammonium groups, among others.

Nanoscale phase domains may also be achieved by providing covalent interactions between the polymeric and ceramic phases. This result can be achieved via a number of known techniques, including the following: (a) providing species with both polymer and ceramic precursor groups and thereafter conducting polymerization and hydrolysis/condensation simultaneously, (b) providing a ceramic sol with polymer precursor groups (e.g., groups that are capable of participation in a polymerization reaction, such as vinyl groups or cyclic ether groups) and thereafter conducting an organic polymerization step, (c) providing polymers with ceramic precursor groups (e.g., groups that are capable of participation in hydrolysis/condensation, such as metal or semi-metal alkoxide groups), followed by hydrolysis/condensation of the precursor groups.

Hybrid polymer-ceramic porous regions may be formed, for example, from hybrid polymer-ceramic fibers, using any suitable fiber-based fabrication technique including, for example, various woven and non-woven techniques. Hybrid polymer-ceramic fibers which have been reported in the literature include poly(vinyl alcohol)/silica fibers, poly(ethylene glycol)/silica fibers, poly(vinyl pyrrolidone)/titania fibers and poly(vinyl acetate)/niobium oxide fibers, among others. See, e.g., C. Shao et al., “Fiber mats of poly(vinyl alcohol)/silica composite via Electrospinning,” Materials Letters 57 (2003) 1579-1584; B. Granqvist et al., “Biodegradable and bioactive hybrid organic-inorganic PEG-siloxane fibers. Preparation and characterization,” Colloid Polym Sci (2004) 282: 495-501; I. S. Chronakis, “Novel nanocomposites and nanoceramics based on polymer nanofibers using electrospinning process—A review,” Journal of Materials Processing Technology 167 (2005) 283-293.

In the above described techniques, the porous layers are porous as applied. However, as previously noted, layers may be provided that are initially non-porous but which are rendered porous prior to insertion/implantation into a subject (and more typically, prior to packaging), or they may be adapted to become porous at a specific time after insertion or implantation within a patient.

For example, an organic-inorganic hybrid layer such as a polymer-ceramic hybrid layer may first be formed using known techniques (e.g., sol-gel based techniques), followed by removal of the organic portion of the layer, leaving behind a porous inorganic layer. For example, the organic portion of a hybrid layer may be removed by subjecting the layer to conditions which are capable of degrading the organic portion, for instance, by heating the hybrid material. If the therapeutic agent is not capable of withstanding the temperatures required for this process step, then the therapeutic agent may be introduced beneath or within the porous layer after the heating step.

As another example, a layer may be designed to become porous at a specific time post insertion/implantation, for example, by including a degradable material (e.g., one of the biodegradable polymers above) into the pores of a slower degrading or biostable material. One specific example of such a layer is a polymer-ceramic hybrid material in which the polymer is biodegradable.

As previously indicated, in accordance with an aspect of the invention, medical devices are provided in which a porous layer, such as those described above, among others, lies over a therapeutic-agent-containing region. Consequently, upon implantation or insertion of the device, therapeutic agent is allowed to diffuse from the underlying therapeutic-agent-containing region, through fluid (e.g., bodily fluid) within the pores of the porous layer, rather than having to diffuse though the solid material making up the porous layer.

“Therapeutic agents”, “pharmaceuticals,” “pharmaceutically active agents”, “drugs” and other related terms may be used interchangeably herein and include genetic therapeutic agents and non-genetic therapeutic agents. Therapeutic agents may be used singly or in combination. Therapeutic agents may be, for example, nonionic or they may be anionic and/or cationic in nature.

Therapeutic agents include, for example, adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, anti-amebic agents, anti-androgen agents, anti-anemic agents, anti-anginal agents, anti-anxiety agents, anti-arthritic agents, anti-asthmatic agents, anti-atherosclerotic agents, antibacterial agents, anticholelithic agents, anticholelithogenic agents, anticholinergic agents, anticoagulants, anticoccidal agents, anticonvulsants, antidepressants, antidiabetic agents, antidiuretics, antidotes, antidyskinetics agents, anti-emetic agents, anti-epileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipoproteinemic agents, antihypertensives, antihypotensives, anti-infective agents, anti-inflammatory agents, antikeratinizing agents, antimicrobial agents, antimigraine agents, antimitotic agents, antimycotic agents, antineoplastic agents, anticancer supplementary potentiating agents, antineutropenic agents, antiobsessional agents, antiparasitic agents, antiparkinsonian drugs, antipneumocystic agents, antiproliferative agents, antiprostatic hypertrophy drugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, cardioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMGCoA reductase inhibitors, immunizing agents, immunomodulators, immunoregulators, immunostimulants, immunosuppressants, impotence therapy adjuncts, keratolytic agents, LHRH agonists, luteolysin agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine Al antagonists, serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, xanthine oxidase inhibitors, and the like.

Exemplary non-genetic therapeutic agents for use in connection with the present invention include the following, among others: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anticoagulants such as D-Phe-Pro-Arg chloromethyl ketone, an RGD peptide-containing compound, heparin, hirudin, antithrombin compounds, platelet receptor antagonists, antithrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, All, AIII, AIV, AV, etc.).

Various preferred non-genetic therapeutic agents include paclitaxel (including particulate forms thereof, for instance, protein-bound paclitaxel particles such as albumin-bound paclitaxel nanoparticles, e.g., ABRAXANE and paclitaxel-polymer conjugates, for example, paclitaxel-poly(glutamic acid) conjugates), rapamycin (sirolimus) and its analogs (e.g., everolimus, tacrolimus, zotarolimus, etc.) as well as sirolimus-polymer conjugates and sirolimus analog-polymer conjugates such as sirolimus-poly(glutamic acid) and everolimus-poly(glutamic acid) conjugates, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, betablockers, bARKct inhibitors, phospholamban inhibitors, Serca 2 gene/protein, imiquimod, human apolioproteins (e.g., AI-AV), growth factors (e.g., VEGF-2), as well a derivatives of the forgoing, among others.

Exemplary genetic therapeutic agents for use in connection with the present invention include anti-sense DNA and RNA as well as DNA coding for the various proteins (as well as the proteins themselves), for example, the following, among others: (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers such as polyvinylpyrrolidone (PVP), SP1017 (SUPRATEK), lipids such as cationic lipids, liposomes, lipoplexes, nanoparticles, or microparticles, with and without targeting sequences such as the protein transduction domain (PTD).

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, coichicine, Epo D, paclitaxel and epothilone), caspase activators, proteasome inhibitors, angiogenesis inhibitors (e.g., endostatin, angiostatin and squalamine), rapamycin (sirolimus) and its analogs (e.g., everolimus, tacrolimus, zotarolimus, etc.), cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference.

A wide range of therapeutic agent loadings can be used in conjunction with the medical devices of the present invention, with the pharmaceutically effective amount being readily determined by those of ordinary skill in the art and ultimately depending, for example, upon the condition to be treated, the nature of the therapeutic agent itself, the tissue into which the medical device is introduced, and so forth.

In some embodiments, the therapeutic-agent-containing region consists essentially of at least one therapeutic agent.

In some embodiments, at least one therapeutic agent is mixed, blended or otherwise commingled with another material, for example, biodegradable organic or inorganic materials, such as one or more of the biodegradable materials described above, among others (e.g., polyester homopolymers and copolymers, polyanhydride homopolymers and copolymers, and/or amino acid based homopolymers and copolymers, among others).

In some embodiments, at least one therapeutic agent (which may optionally be mixed, blended or otherwise commingled with at least one other material such as a biodegradable organic or inorganic material) is provided within the interstices of a porous layer. The porous layer may be, for example, one of those described above, among others. The therapeutic agent (along with any optional commingled material) may be introduced, for example, concurrently with or after the formation of the porous layer. For example, a therapeutic-agent-containing liquid composition (e.g., one containing one or more therapeutic agents, along with any optional species such as one or more biodegradable organic or inorganic materials and/or one or more solvent species, among others) can be injected into the porous layer via micro-needles, or the porous layer can be sprayed with, or dipped into, the therapeutic-agent-containing liquid composition, thereby introducing the therapeutic agent into the interstices of the porous layer. If desired, the resulting structure can be optionally ablated (e.g., by laser ablation, etc.) to expose the porous surface.

In certain embodiments, the therapeutic-agent-containing region constitutes the bulk of a medical device (e.g., a stent) or a portion thereof (e.g., one or both ends of a medical device such as a stent, a distinct component of a multi-component device, etc.). In these embodiments, for example, the entire therapeutic-agent-containing region may be biodegradable (e.g., one or more therapeutic agents may be commingled with one or more biodegradable materials), or only a portion of the therapeutic-agent-containing region may be biodegradable (e.g., in the form of a biodegradable, therapeutic-agent-containing material filling the interstices of a biostable porous layer).

In certain other embodiments, the therapeutic-agent-containing region is disposed between a substrate and the porous layer, which substrate may constitute, for example, the bulk of an entire medical device or a portion thereof. The substrate region may be selected, for example, from suitable biostable and biodegradable members of the organic, inorganic, and organic-inorganic hybrid materials described above (e.g., biostable and biodegradable metals and metal alloys, biostable and biodegradable polymers and polymer blends, biostable and biodegradable ceramic materials, and biostable and biodegradable polymer-ceramic hybrid materials, among others).

In certain embodiments, one or more optional additional layers may be provided in the medical devices of the invention. For example, an optional additional layer such as a biodegradable organic material, inorganic material or organic-inorganic hybrid material (e.g., a biodegradable polymeric layer, metallic or ceramic layer, among others) may be provided between the therapeutic-agent-containing region and the exterior of the device to delay release. For example, the additional biodegradable layer may be located between the therapeutic-agent-containing region and the porous layer, or it may be located outside of the porous layer.

In various embodiments described above, the therapeutic component is able to move more or less perpendicularly with respect to the substrate in order to be released into the surrounding environment. In certain other embodiments, portions (but not all) of the therapeutic-agent-containing region are covered with a non-porous layer (e.g., a non-porous biostable layer), such that the therapeutic component is forced to initially travel a certain distance parallel to the substrate surface in order to reach the porous upper layer.

As is clear from the above discussion, a variety of structures can be formed in accordance with the present invention, several specific examples of which will now be discussed in conjunction with the drawings. Although tubular medical devices such as stents are specifically disclosed, the present invention is applicable to a wide variety of medical devices as noted above.

FIG. 2 is a stent body 100, analogous in design to that described in more detail in U.S. Patent Pub. No. 2004/0181276, and comprises various struts 100s. Unlike the stent of U.S. Patent Pub. No. 2004/0181276, however, stent body 100 is constructed to release therapeutic agent in accordance with the present invention, and it thus includes a porous layer which is disposed over a drug containing region. In this regard, schematic cross-sectional views taken along line a-a of FIG. 2 are illustrated in FIGS. 3A-3D, in accordance with four alternative embodiments of the present invention. (It will be clear to those of ordinary skill in the art that other constructions in accordance with the present invention are possible and that the constructions of FIGS. 3A-3D may be employed in numerous medical devices other than stents.)

In accordance with an embodiment of the invention illustrated schematically in cross-section in FIG. 3A, a biostable porous layer 160 is disposed over a biodegradable therapeutic-agent-containing layer 150 (e.g., one containing one or more therapeutic agents as well as one or more biodegradable materials such as those listed above), which is in turn disposed over a biostable or biodegradable substrate 110.

In accordance with another embodiment of the invention illustrated schematically in cross-section in FIG. 3B, a biostable porous layer 160 is provided over a therapeutic-agent-containing layer 152 that is partially biostable and partially biodegradable (e.g., a biostable porous layer whose interstices are at least partially filled with a material that contains one or more therapeutic agents as well as one or more biodegradable materials). The therapeutic-agent-containing layer 152 is in turn disposed over a biostable or biodegradable substrate 110.

In accordance with another embodiment of the invention illustrated schematically in cross-section in FIG. 3C, a biodegradable porous layer 162 is provided over a therapeutic-agent-containing layer 152 that is partially biostable and partially biodegradable (e.g., a biostable porous layer whose interstices are at least partially filled with a material that contains one or more therapeutic agents and one or more biodegradable materials). The therapeutic-agent-containing layer 152 is in turn disposed over a biostable or biodegradable substrate 110.

In accordance with yet another embodiment of the invention illustrated schematically in cross-section in FIG. 3D, a biostable porous layer 160 is provided over a biodegradable layer 170 (e.g., one containing one or more biodegradable materials), which is disposed over a therapeutic-agent-containing layer 154 (e.g., one consisting essentially of one or more therapeutic agents or one containing one or more therapeutic agents as well as one or more biodegradable materials), which is in turn provided over a biostable or biodegradable substrate 110.

Potential benefits of each of the structures of FIGS. 3A-3D include one or more of the following, among others: (a) therapeutic agent is readily eluted from the medical device (after dissolution of biodegradable layer 170, where present), (b) where the substrate 110 is bioadverse, a porous barrier (e.g., a porous layer 160, a porous biostable remnant of therapeutic-agent-containing layer 152, or a combination of both) surrounds the substrate 110, reducing or eliminating the adverse affects of the same, (c) where the substrate 110 is biodegradable, a porous barrier (e.g., a porous layer 160, a porous biostable remnant of therapeutic-agent-containing layer 152, or a combination of both) surrounds the substrate 110, preventing large substrate fragments from being released into the body, and (d) a porous layer is provided which may, in certain embodiments, facilitate tissue attachment and/or growth.

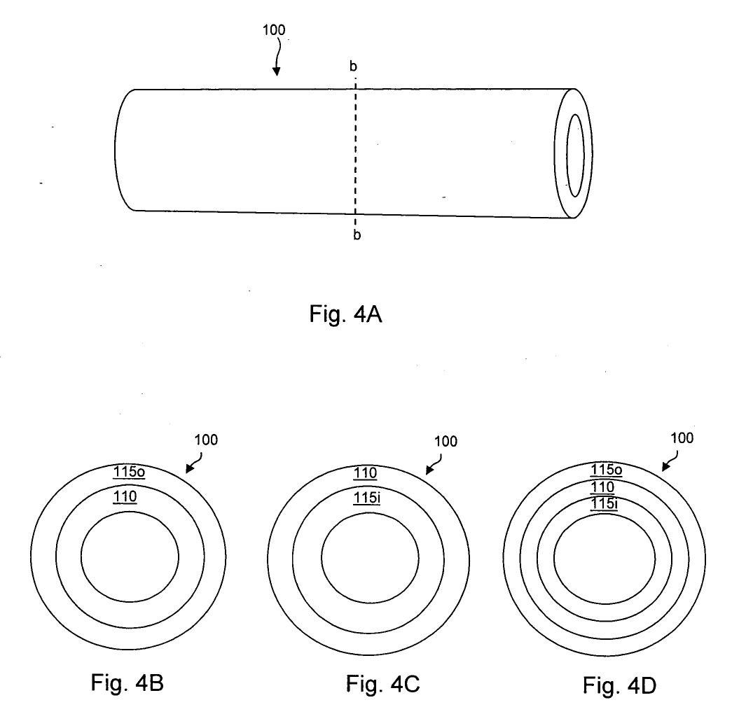

Another example of a medical device in accordance with the present invention is a tubular medical device 100 such as that shown in perspective view in FIG. 4A. Alternative cross-sections taken along line b-b of FIG. 4A are illustrated in FIGS. 4B, 4C and 4D, in accordance with various embodiments of the invention. In FIG. 4B a biostable or biodegradable substrate 110 is provided with an outer region 115o, in accordance with an embodiment of the invention, whereas the inner surface of the substrate 110 remains bare. In FIG. 4C, a biostable or biodegradable substrate 110 is provided with an inner region 115i, in accordance with an embodiment of the invention, whereas the outer surface of the substrate 110 remains bare. In FIG. 4D, inner and outer surfaces of a biostable or biodegradable substrate 110 are provided with an inner region 115i and an outer region 115o, in accordance with an embodiment of the invention.

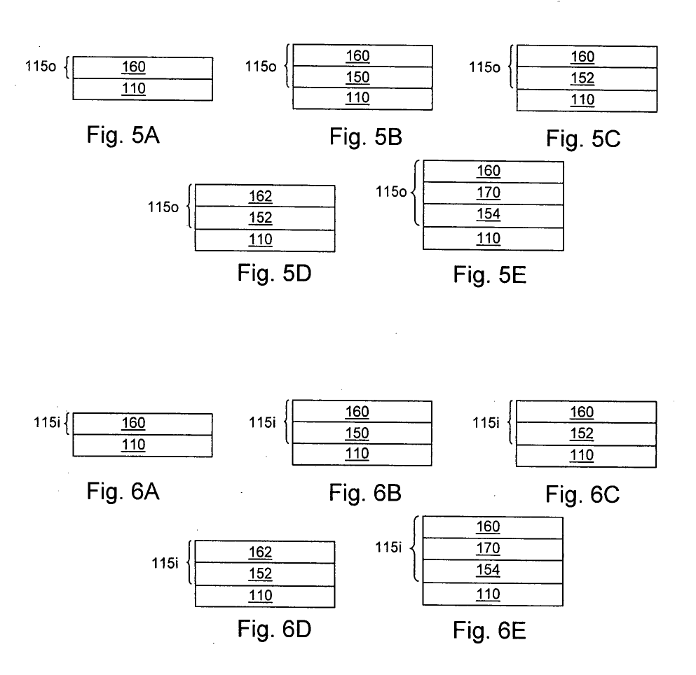

Outer and inner regions 115o and 115i may each contain one or more layers. For example, these regions may be independently selected from the constructions schematically illustrated in FIGS. 5A-5E and in 6A-6E, among other possibilities.

As shown in FIGS. 5A and 6A, the outer region 115o and/or inner region 115i may be in the form of a biostable porous layer 160 adjacent substrate 110. Potential benefits of such a structure include one or more of the following, among others: (a) where the substrate 110 is a least partially biodegradable and contains a therapeutic agent, therapeutic agent is readily eluted from the inner/and or outer surfaces of the medical device, (b) where the substrate 110 is bioadverse, a porous barrier is disposed over the inner/and or outer surfaces of the substrate 110, (c) where the substrate 110 is biodegradable, a porous barrier may surround the substrate 110, preventing, for example, large fragments from being released into the body, and (d) a porous layer is provided, which may facilitate tissue attachment and/or growth in some embodiments.

As shown in FIGS. 5B and 6B, the outer region 115o and/or the inner region 115i may comprise a biodegradable therapeutic-agent-containing layer 150 (e.g., one containing one or more therapeutic agents as well as one or more biodegradable materials) and a biostable porous layer 160, wherein the biodegradable therapeutic-agent-containing layer 150 is disposed between the substrate 110 and the biostable porous layer 160.

As shown in FIGS. 5C and 6C, the outer region 115o and/or inner region 115i may comprise a therapeutic-agent-containing layer 152 that is partially biostable and partially biodegradable (e.g., a biostable porous layer whose interstices are at least partially filled with a material that contains one or more therapeutic agents as well as one or more biodegradable materials) and a biostable porous layer 160, wherein the therapeutic-agent-containing layer 152 is disposed between the substrate 110 and the biostable porous layer 160.

As shown in FIGS. 5D and 6D, the outer region 115o and/or inner region 115i may comprise a therapeutic-agent-containing layer 152 that is partially biostable and partially biodegradable (e.g., a biostable porous layer whose interstices are at least partially filled with a material that contains one or more therapeutic agents as well as one or more biodegradable materials) and a biodegradable porous layer 162, wherein the therapeutic-agent-containing layer 152 is disposed between the substrate 110 and the biodegradable porous layer 162.

As shown in FIGS. 5E and 6E, the outer region 115o and/or inner region 115i may comprise a biostable porous layer 160, an optional biodegradable layer 170 (e.g., one containing one or more one or more biodegradable materials), and a therapeutic-agent-containing layer 154 (e.g., one consisting essentially of one or more therapeutic agents or one containing one or more therapeutic agents as well as one or more biodegradable materials).

Potential benefits of each of the structures of FIGS. 5B-5E and 6B-6E include one or more of the following, among others: (a) therapeutic agent is readily eluted from the inner and/or outer surfaces of the device 100 (after dissolution of biodegradable layer 170, if present), (b) where the substrate 110 is bioadverse, a porous barrier (e.g., a porous layer 160, a porous biostable remnant of therapeutic-agent-containing layer 152, or a combination of both) is disposed over the inner and/or outer surfaces of the substrate 110, reducing or eliminating the adverse affects of the same, (c) where the substrate 110 is biodegradable, a porous barrier (e.g., a porous layer 160, a porous biostable remnant of therapeutic-agent-containing layer 152, or a combination of both) may surround the substrate 110, preventing large substrate fragments from being released into the body, and (d) a porous layer is provided which may facilitate tissue attachment and/or growth, in some embodiments.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising a substrate region, a porous layer disposed over said substrate region, a therapeutic agent disposed beneath said porous layer, and a biodegradable material disposed beneath said porous layer that regulates the release of said therapeutic agent from said medical device into a subject upon implantation or insertion of said device into said subject.

2. The medical device of claim 1, wherein said porous layer is biostable.

3. The medical device of claim 1, wherein said porous layer is a polymeric layer.

4. The medical device of claim 1, wherein said porous layer is a ceramic layer.

5. The medical device of claim 1, wherein said porous layer is a polymer-ceramic hybrid layer.

6. The medical device of claim 1, wherein said porous layer is a metallic layer.

7. The medical device of claim 1, wherein said porous layer is comprises fibers.

8. The medical device of claim 7, wherein said fibers are interconnected.

9. The medical device of claim 7, wherein fibers have diameters between 20 and 5000 nm.

10. The medical device of claim 1, wherein said porous layer comprises interconnected particles.

11. The medical device of claim 10, wherein said particles have diameters between 20 and 5000 nm.

12. The medical device of claim 1, wherein said porous layer is an electrostatically deposited layer.

13. The medical device of claim 1, wherein said porous layer is deposited using a field-injection-based electrospray technique.

14. The medical device of claim 1, wherein said substrate region is a polymeric substrate region.

15. The medical device of claim 1, wherein said substrate region is a ceramic substrate region.

16. The medical device of claim 1, wherein said substrate region is a metallic substrate region.

17. The medical device of claim 1, wherein said substrate region is a bioadverse substrate region.

18. The medical device of claim 1, wherein said substrate region is a biostable substrate region.

19. The medical device of claim 1, wherein said substrate region is a biodegradable substrate region.

20. The medical device of claim 1, wherein said substrate region comprises said therapeutic agent and said biodegradable material.

21. The medical device of claim 1, wherein said medical device comprises a therapeutic-agent-containing layer that comprises said therapeutic agent and said biodegradable material, and wherein said therapeutic-agent-containing layer is disposed between said substrate and said porous layer.

22. The medical device of claim 21, wherein said therapeutic-agent-containing layer is completely biodegradable.

23. The medical device of claim 21, wherein said therapeutic-agent-containing layer is partially biodegradable.

24. The medical device of claim 23, wherein said partially biodegradable therapeutic-agent-containing layer comprises a biostable porous portion and a therapeutic-agent-containing portion comprising said therapeutic agent and said biodegradable material disposed within the interstices of said biostable porous portion.

25. The medical device of claim 24, wherein said porous layer is biodegradable.

26. The medical device of claim 1, wherein said medical device comprises a therapeutic-agent-containing layer comprising said therapeutic agent disposed over said substrate, wherein said medical device comprises a biodegradable layer comprising said biodegradable material disposed over said therapeutic-agent-containing layer, and wherein said porous layer is disposed over said biodegradable layer.

27. The medical device of claim 26, wherein said biodegradable material is a biodegradable polymer.

28. The medical device of claim 1, wherein said porous region surrounds said substrate region, said therapeutic agent, and said biodegradable material.

29. The medical device of claim 1, wherein said substrate region has inner and outer surfaces and wherein said porous layer is disposed over one or both of said surfaces.

30. The medical device of claim 1, wherein said substrate region has inner and outer surfaces, wherein a first porous layer is disposed over said inner surface, and wherein a second porous layer is disposed over said outer surface, wherein said first and second porous layers may be formed from the same or different materials.

31. The medical device of claim 30, comprising (a) a first therapeutic agent and a first biodegradable material between said first porous layer and said inner surface, and (b) a second therapeutic agent and a second biodegradable material between said second porous layer and said outer surface, wherein said first and second therapeutic agents may the same or different and wherein said first and second biodegradable materials may be the same or different.

32. A method of forming a medical device comprising depositing a porous layer over a therapeutic-agent-containing region using a field-injection-based electrospray technique.

33. The medical device of claim 1, further comprising an additional therapeutic agent.

34. The medical device of claim 33, wherein said additional therapeutic agent is disposed within said porous layer.

35. The medical device of claim 1, wherein said therapeutic agent is selected from paclitaxel, paclitaxel-polymer conjugates, everolimus, everolimus-polymer conjugates, and combinations thereof.
